(19) United States
(12) Patent Application Publication
(10) Pub. No.: US 2014/0377180 A1
(43) Pub. Date: Dec. 25, 2014

(54) PHOTOACOUSTIC CONTRAST AGENT HAVING LIPID PARTICLE CONTAINING SILICON NAPHTHALOCYANINE ANALOG

(71) Applicant: CANON KABUSHIKI KAISHA, Tokyo (JP)

(72) Inventors: Fumiko Tomatsu, Yokohama-shi (JP); Tatsuki Fukui, Yokohama-shi (JP); Daisuke Sasaguri, Yokohama-shi (JP); Mayuko Kishi, Machida-shi (JP); Kouichi Kato, Kyoto-shi (JP); Satoshi Yuasa, Yokohama-shi (JP)

(21) Appl. No.: 14/300,433

(22) Filed: Jun. 10, 2014

(30) Foreign Application Priority Data
Jun. 24, 2013 (JP) 2013-131878

(51) Int. Cl. A61K 49/22

(52) U.S. Cl. CPC A61K 49/22
USPC 424/9.1

(57) ABSTRACT

A conventional silicon naphthalocyanine-containing liposome is required to incorporate a dye dispersed therein for use in photodynamic therapy, and is thus low in dye content and also low in photoacoustic signal. According to a lipid particle containing a silicon naphthalocyanine analog, in which the ratio A/B of an absorption coefficient A at a first absorption local maximum to an absorption coefficient B at a second absorption local maximum in a range from 700 nm to 800 nm is 5.0 or less, the dye content in the lipid particle can be increased and the photoacoustic signal intensity can be improved.

### BACKGROUND OF THE INVENTION

1. Field of the Invention

The present invention relates to a photoacoustic contrast agent having a lipid particle containing a silicon naphthalocyanine analog.

2. Description of the Related Art

In recent years, a photoacoustic imaging method has attracted attention as a method for non-invasively visualizing information on the inside of a living body.

In the measurement using the photoacoustic imaging method, an object to be measured is irradiated with light, and the intensity and the time of occurrence of a photoacoustic signal here generated by a substance (light absorber) that absorbs the light inside of the object to be measured is measured to thereby enable to compute and visualize the substance distribution of the inside of the object to be measured.

For the light absorber, one that absorbs light in a living body to emit acoustic wave or fluorescence can be suitably used. For example, blood vessels or malignant tumors in a human body can be used for the light absorber, and the acoustic wave emitted from the light absorber can be measured. Furthermore, a dye that absorbs light in a near-infrared wavelength region, or the like, can also be administered to a body and utilized as a contrast agent. Since light in a near-infrared wavelength region has a small influence during irradiation to a human body and has a high permeability to a living body, the dye that absorbs light in a near-infrared wavelength region can be suitably used as a contrast agent in the photoacoustic imaging method. In the present specification, the dye is defined as a compound that can absorb light having a wavelength included in a range from 600 nm to 1300 nm.

The contrast agent, in order to effectively amplify the signal intensity (the intensity of acoustic wave or fluorescence), is demanded to have an increased dye content by accumulation of the dye on a particle, a micelle, a polymer micelle, a liposome, or the like (collectively referred to as a particle or the like), resulting in the increase in absorption efficiency of irradiation energy.

A liposome containing silicon naphthalocyanine has been heretofore reported (Br. J. Cancer (1990), 62, 966-970). On the other hand, it is known for phthalocyanine and naphthalocyanine that the absorption spectrum changes between a monomer and an aggregate (Ciba Foundation symposium 146). In British Journal of Cancer (1995) 71, 727-732, the degree of aggregation of phthalocyanine incorporated in a liposome is calculated from the ratio between absorption coefficients at two absorption local maximums.

The liposome disclosed in Br. J. Cancer (1990), 62, 966-970 is for photodynamic therapy (PDT). With respect to the liposome in Br. J. Cancer (1990), 62, 966-970, in order to generate active oxygen, silicon naphthalocyanine is required to be encapsulated in the liposome in the state of being dispersed (monomer) so as not to be aggregated. As a result, the liposome has the problem of having a low dye content and a low photoacoustic signal intensity.

Then, an object of the present invention is to provide a lipid particle (for example, a liposome) having a high dye content.

### SUMMARY OF THE INVENTION

The inventors of the present application have made intensive studies, and as a result, have found that a lipid particle containing a silicon naphthalocyanine analog, in which an absorption coefficient A at a wavelength a as a first absorption local maximum and an absorption coefficient B at a wavelength b as a second absorption local maximum shown in the absorption spectrum of the particle are as low as 5 or less, has a high photoacoustic contrast effect, completing the present invention.

That is, the photoacoustic contrast agent according to the present invention is a photoacoustic contrast agent having a lipid particle containing a silicon naphthalocyanine analog, wherein an absorption coefficient A that is the largest value and an absorption coefficient B that is the second largest value and a local maximum value in a wavelength region of 700 nm to 800 nm, of the lipid particle, satisfy the following expression (1).

0<A/B≦5.0 (1)

Further features of the present invention will become apparent from the following description of exemplary embodiments with reference to the attached drawings.

### BRIEF DESCRIPTION OF THE DRAWINGS

FIG. 1 illustrates a relationship diagram between a dye content and an absorption coefficient ratio in Example 1 of the present invention.

FIG. 2 illustrates a relationship diagram between a dye content and a photoacoustic signal intensity per 100-nm-equivalent particle in Example 1 of the present invention.

### DESCRIPTION OF THE EMBODIMENTS

Preferred embodiments of the present invention will now be described in detail in accordance with the accompanying drawings.

A photoacoustic contrast agent according to the present embodiment has a lipid particle containing a silicon naphthalocyanine analog. Then, an absorption coefficient A that is the largest value and an absorption coefficient B that is the second largest value and a local maximum value in a wavelength region of 700 nm to 800 nm, of the lipid particle, satisfy the following expression (1). 0<A/B≦5.0 (1)

Herein, the silicon naphthalocyanine analog has a different absorption spectrum in a wavelength region of 700 nm to 800 nm between the case of being present as a monomer and the case of being present as an aggregate in a solvent. Accordingly, a mixture of the monomer and aggregate of the silicon naphthalocyanine analog has a plurality of absorption local maximums in a wavelength region of 700 nm to 800 nm. Among them, the absorption local maximum wavelength at the absorption coefficient A that is the largest value is herein referred to as “wavelength a”, and the absorption local maximum wavelength at the absorption coefficient B that is the second largest value and a local maximum value is herein referred to as “wavelength b”.

While a and b differ depending on solvents and measurement conditions, a can be in a range of more than 750 nm and 800 nm or less, and b can be in a range of 700 nm or more and 750 nm or less.

Accordingly, the above expression (1) defines the ratio of the aggregate to the monomer of the silicon naphthalocyanine analog in the lipid particle. When the above expression (1) is satisfied, the amount of the silicon naphthalocyanine analog can be large to allow the silicon naphthalocyanine analog to be incorporated into the lipid particle in a large amount. As a result, the photoacoustic contrast agent according to the present embodiment has a large molar absorbance coefficient and a high photoacoustic intensity.

In the present embodiment, the absorption coefficient A and the absorption coefficient B can satisfy the following expression (2).

0<A/B≦2.5 (2)

(Silicon Naphthalocyanine Analog)

The silicon naphthalocyanine analog according to the present embodiment may be any analog as long as the analog has a naphthalocyanine backbone and a silicon compound in the center. Since the naphthalocyanine backbone is hydrophobic, silicon naphthalocyanines having the naphthalocyanine backbone or derivatives thereof plurally aggregate easily by a hydrophobic interaction. The silicon naphthalocyanines or derivatives thereof that plurally aggregate are higher in hydrophobicity. Therefore, when the lipid particle according to the present embodiment is placed in an aqueous solution such as serum, the silicon naphthalocyanines or derivatives thereof are hardly leaked out of the particle.

The silicon naphthalocyanine analog has absorption in a near-infrared wavelength of 600 nm to 900 nm, excellent in living body permeability. Since the lipid particle according to the present embodiment contains the silicon naphthalocyanine analog, the particle can be safe upon irradiation to a living body, and can absorb wavelength having a wavelength in a near-infrared wavelength region (near-infrared wavelength region of 600 nm to 900 nm) and having a relatively high permeability to a living body. In the present embodiment, the structure of the silicon naphthalocyanine analog is represented by the following chemical formula (1).

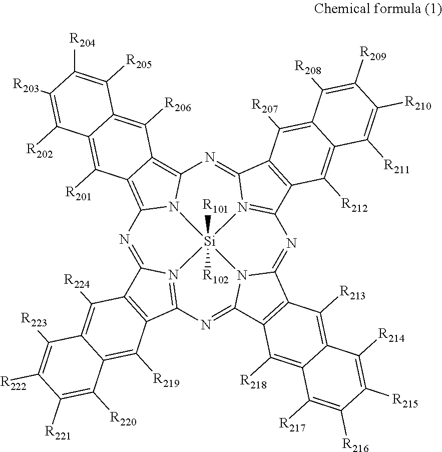

wherein R₂₀₁, R₂₀₂, R₂₀₃, R₂₀₄, R₂₀₅, R₂₀₆, R₂₀₇, R₂₀₈, R₂₀₉, R₂₁₀, R₂₁₁, R₂₁₂, R₂₁₃, R₂₁₄, R₂₁₅, R₂₁₆, R₂₁₇, R₂₁₈, R₂₁₉, R₂₂₀, R₂₂₁, R₂₂₂, R₂₂₃ and R₂₂₄ may be each the same or different, and each represent a hydrogen atom, a halogen atom, an acetoxy group, an amino group, a nitro group, a cyano group, or an alkyl group having 1 to 18 carbon atoms or an aromatic group that is unsubstituted or substituted with one or more functional groups selected from the group consisting of a halogen atom, an acetoxy group, an amino group, a nitro group, a cyano group or an alkyl group having 1 to 18 carbon atoms.

In addition, R₁₀₁ and R₁₀₂ may be each the same or different, and each represent —OH, —OR₁₁, —OCOR₁₂, —OSi (—R₁₃)(—R₁₄)(—R₁₅), a halogen atom, an acetoxy group, an amino group, a nitro group, a cyano group, or an alkyl group having 1 to 18 carbon atoms or an aromatic group that is unsubstituted or substituted with one or more functional groups selected from the group consisting of a halogen atom, an acetoxy group, an amino group, a nitro group, a cyano group, or an alkyl group having 1 to 18 carbon atoms.

Herein, R₁₁, R₁₂, R₁₃, R₁₄ and R₁₅ may be each the same or different, and each represent one that is unsubstituted or substituted with one or more functional groups selected from the group consisting of a halogen atom, an acetoxy group, an amino group, a nitro group, a cyano group, or an alkyl group having 1 to 18 carbon atoms.

Specific examples of the silicon naphthalocyanine analog can include silicon 2,3-naphthalocyanine bis(trihexylsilyloxide) (Silicon 2,3-naphthalocyanine bis (trihexylsilyloxide)).

(Phospholipid)

The lipid particle in the photoacoustic contrast agent of the present embodiment can include phospholipid. While examples of the phospholipid can include synthesized distearoylphosphatidylcholine (DSPC), other alkyl or alkenyl derivatives of synthesized phosphatidic acid (PA) or the like can also be used, and at least one selected from the group consisting of, for example, dimyristoylphosphatidylcholine (DMPC), dipalmitoylphosphatidylcholine (DPPC), dioleylphosphatidylcholine (DOPC), distearoylphosphatidylserine (DSPS), distearoylphosphatidylglycerol (DSPG) and dipalmitoylphosphatidic acid (DPPA) can be used.

Other phospholipids include soybean or egg-yolk lecithin, lysolecithin, or derivatives of hydrogenated products or hydroxides thereof, or semisynthetic phosphatidylcholine, phosphatidylserine (PS), phosphatidylethanolamine, phosphatidylglycerol (PG), phosphatidylinositol (PI) or sphingomyelin.

(Polyethyleneglycol)

The lipid particle according to the present embodiment can have a polyethyleneglycol chain introduced to the lipid membrane surface of the lipid particle. Application examples of the lipid particle of the present embodiment include a tumor contrast agent. In order to exert the EPR (Enhanced permeability and retention, the enhancement in permeability in tumor blood vessels and retention in tumors) effect proposed as the principle of passive targeting to tumors, the contrast agent is demanded to have a high retentivity in blood. Polyethyleneglycol has a suppressed interaction with a protein in blood, such as complement, to thereby be hardly phagocytized by reticuloendothelial cells of liver or the like, enabling the retentivity in blood of the liposome to be improved. Therefore, it is very advantageous to introduce polyethyleneglycol to the lipid particle of the present embodiment.

The molecular weight of polyethyleneglycol and the introduction rate thereof to the lipid particle can be appropriately changed to thereby regulate the function of polyethyleneglycol. Polyethyleneglycol having a molecular weight of 500 or more and 200000 or less can be used, and in particular, polyethyleneglycol having a molecular weight of 2000 or more and 100000 or less is suitable. In addition, the introduction rate of polyethyleneglycol to the lipid particle, that is, the proportion of polyethyleneglycol in the lipid particle is preferably 0.001% by mol or more and 50% by mol or less, further preferably 0.01% by mol or more and 30% by mol or less, and more preferably 0.1% by mol or more and 10% by mol or less, relative to the lipid constituting the lipid particle.

For the method for introducing polyethyleneglycol to the lipid particle, a known technique can be utilized. An example can be a method for producing a lipid particle with polyethyleneglycol-bound phospholipid or the like being included in phospholipids serving as a lipid particle raw material in advance. Examples of the polyethyleneglycol-bound phospholipid can include polyethyleneglycol-bound phospholipids such as 1,2-distearoyl-sn-glycero-3-phosphoethanolamine-N-[carboxy(polyethylene glycol)] (DSPE-PEG-OH), Poly(oxy-1,2-ethanediyl), α-[7-hydroxy-7-oxido-13-oxo-10-[(1-oxooctadecyl)oxy]-6,8,12-trioxa-3-aza-7-phosphatriacont-1-yl]-ω-methoxy-(DSPE-PEG-OMe), N-(aminopropyl polyethyleneglycol)-carbamyl distearoylphosphatidyl-ethanolamine (DSPE-PEG-NH2), 3-(N-succinimidyloxyglutaryl)aminopropyl polyethyleneglycol-carbamyl distearoylphosphatidyl-ethanolamine (DSPE-PEG-NHS), N-(3-maleimide-1-oxopropyl)aminopropyl polyethyleneglycol-carbamyl distearoylphosphatidyl-ethanolamine (DSPE-PEG-MAL), SUNBRIGHT (registered trademark) DSPE-020-PA, SUNBRIGHT (registered trademark) DSPE-020-CN, SUNBRIGHT (registered trademark) DSPE-050-CN, Methoxyl PEG DSPE (Mw10000) and Methoxyl PEG DSPE (Mw20000). Among them, examples of polyethyleneglycol phospholipid can include SUNBRIGHT (registered trademark) DSPE-020-CN.

(Lipid Particle)

In the present embodiment, the lipid particle is a lipid particle constituted of at least a lipid such as phospholipid, and also includes a lipid vesicle or a liposome. While a liposome generally means a lipid vesicle constituted of one or multi layers of a double membrane mainly constituted of phospholipid, the lipid particle according to the present embodiment is not limited to such a liposome, and includes all lipid particles constituted of at least lipid such as phospholipid and also a lipid particle that can be dispersed in a dispersion medium even if silicon naphthalocyanine enters a lipid membrane to disturb the organization of the lipid membrane. The lipid particle may also include, as a constituent component, a lipid, a glycolipid, a sterol derivative, a lipid derivative, and a combination thereof. The lipid particle may also be constituted of a mixture of different lipids. As the lipid derivative, for example, polyethyleneglycol-bound phospholipid can also be used. For the method for preparing the lipid particle, a conventionally known method for preparing a liposome can be utilized, and can be appropriately selected in order to provide a lipid particle having desired physical properties. The type, amount and the like of the lipid can be appropriately selected depending on the application of the lipid particle. For example, the type of the lipid, the amount of the lipid, the ratio thereof, and the charge of the lipid can be considered to thereby control the particle size of the lipid particle and the surface potential.

As a constituent material other than the lipid, other materials can also be added, if necessary. Examples include cholesterol acting as a membrane stabilizer, glycols such as ethylene glycol, phosphoric acid dialkyl esters to be added for charge control, and aliphatic amines such as stearylamine.

The lipid particle according to the present embodiment can be prepared by a known method for manufacturing a liposome. A known technique is described in pages 33 to 37 in “Riposomu Ouyou no Shin-tenkai (New development of application of liposomes)”, Kazunari Akiyoshi and Kaoru Tsujii, ed., NTS, published on Jun. 1, 2005. Examples include a Bangham's method (a simple hydration method, a sonication method, and an extrusion method), a pH gradient (remote loading) method and a counter ion concentration gradient method, a freeze-thaw method, a reverse phase evaporation method, a mechanochemical method, a supercritical carbon dioxide method and a film loading method, and also a method using a commercially available hollow liposome. A liposome prepared by any of the known methods can be provided to the lipid particle of the present embodiment.

(Method for Preparing Liposome)

One example of a method for manufacturing a silicon naphthalocyanine-containing lipid particle of the present embodiment can be one according to the method for producing a liposome by a Bangham's method. That is, a method can be used which includes dissolving a raw material for a liposome, such as phospholipid, and a high concentration of silicon naphthalocyanine in an organic solvent for mixing, removing the organic solvent under reduced pressure to dry the lipid and silicon naphthalocyanine for solidification, and dispersing the lipid and silicon naphthalocyanine in an aqueous medium for homogenization by ultrasonic irradiation to thereby form a liposome.

(Lipid Particle Size)

For the lipid particle according to the present embodiment, various ones having different sizes, including small ones of several tens nanometer and large ones of several micrometer, can be used as in the case of a general liposome. In fact, the size of the lipid particle and the distribution of the size are very important in terms of a tumor contrast agent as one application example of the lipid particle of the present embodiment, and are closely associated with the retentivity in blood and the delivery efficiency to a target tissue. Accordingly, the average particle size of the lipid particle can be in particular 20 to 200 nm. The particle size can be measured by electron microscope observation or a particle size measurement method based on a dynamic light scattering method.

(Contrast Agent)

Since the lipid particle according to the present embodiment incorporates silicon naphthalocyanine and absorbs near-infrared light to generate acoustic wave, the lipid particle can be used as a contrast agent for photoacoustic imaging. In addition, since the lipid particle according to the present embodiment is colored in dark green, the lipid particle can also be used as a contrast agent for visual detection.

In the present specification, the “contrast agent” is mainly defined as a substance that is present in a specimen, and that can generate a contrast difference between a tissue or molecule to be observed and a tissue or molecule present in the periphery thereof to improve the detection sensitivity of information on the shape or position of the tissue or molecule to be observed. The “photoacoustic imaging” means imaging of the above-described tissue or molecule by a photoacoustic signal detection apparatus or the like.

The contrast agent including as a main component the lipid particle according to the present embodiment may have a pharmacologically acceptable additive. Examples of the pharmacologically acceptable additive include a tonicity agent, a pH adjuster and a stabilizer, for example, sugars such as sucrose and glucose or polyhydric alcohols such as glycerin or propylene glycol. The additive can be used as a mixture of the contrast agent and an arbitrarily additive before being administered to a living body.

The imaging method using the contrast agent including as a main component the lipid particle according to the present embodiment has a step of administering the contrast agent to a subject, a step of accumulating the contrast agent in a target tissue, and a step of detecting the contrast agent present in the target tissue. The method for detecting the contrast agent includes a direct observation method on gross, a near-infrared fluorescence method and a photoacoustic method.

One example of the photoacoustic imaging method according to the present embodiment is as follows. That is, the contrast agent including as a main component the lipid particle according to the present embodiment is administered to a specimen. Herein, the specimen is not particularly limited, and may be mammals such as human, or other experimental animals and pet animals, or the like. After the contrast agent is administered, the specimen or the like is irradiated with laser pulse light in a near-infrared wavelength region. Then, the photoacoustic signal (acoustic wave) from the contrast agent is detected by an acoustic wave detector, for example, a piezoelectric transducer, and transduced to an electric signal. Based on the electric signal obtained from the acoustic wave detector, the position and size or the optical property value distribution such as light absorbance coefficient, of an absorber in the specimen or the like, can be calculated. One application example of the contrast agent including as a main component the lipid particle according to the present embodiment is to detect tumors.

### EXAMPLES

Hereinafter, specific reagents, reaction conditions and the like for use in producing the lipid particle according to the present embodiment are listed in Examples, but such reagents, reaction conditions and the like can be changed and such changes are encompassed within the scope of the present invention. Accordingly, the following Examples are intended to assist the understanding of the present invention, and do not limit the scope of the present invention at all.

(Recovery Method)

A centrifugation operation was performed using a high speed refrigerated microcentrifuge (manufactured by Tomy Seiko Co., Ltd., MX-300).

(Analysis Method)

Particle size measurement was performed using a dynamic light scattering analysis apparatus (manufactured by Otsuka Electronics Co., Ltd., ELSZ-2).

The measurement was performed using a semiconductor laser as a light source, and the value of a cumulant diameter was adopted as a particle size.
Absorbance measurement was performed using a UV-VIS-NIR measurement apparatus (manufactured by PerkinElmer Co., Ltd., Lambda Bio 40).

(Calculation Method of Dye Content)

Dye quantification of a particle dispersion was performed to calculate the dye amount included in the dispersion . . . . (a)

The particle dispersion was lyophilized to thereby calculate the weight of a solid component included in the dispersion . . . . (b)
The dye amount determined in (a) was divided by the weight of a solid component determined in (b) to thereby calculate the dye content.

(Calculation of Absorption Coefficient Ratio)

Absorbance measurement was performed to determine an absorption coefficient A′ at a wavelength a, an absorption coefficient B′ at a wavelength b, and an absorption coefficient C at 600 nm. The absorption coefficient ratio was calculated by dividing an absorption coefficient A by an absorption coefficient B, wherein the absorption coefficients A and B were obtained by subtracting the absorption coefficient C from the absorption coefficient A′ and subtracting the absorption coefficient C from the absorption coefficient B′ for baseline correction, respectively.

(Measurement Method of Photoacoustic Signal Intensity)

Measurement of the photoacoustic signal intensity was as follows: a sample vessel placed in ultrapure water was irradiated with pulse laser light, and the intensity of a photoacoustic signal generated from the sample in the vessel was detected using a piezoelectric element, amplified by a high-speed pre-amplifier, and then acquired by a digital oscilloscope. Specific conditions were as follows. A titanium sapphire laser (LT-2211-PC, manufactured by Lotis TII) was used as a light source. The wavelength was 780 nm, the energy density was about 10 to 20 mJ/cm², the pulse width was about 20 nanoseconds, and the pulse repetition frequency was 10 Hz. For the piezoelectric element for detecting the photoacoustic signal, a non-focusing type ultrasonic wave transducer (V303, manufactured by Panametrics-NDT Ltd.) having an element diameter of 1.27 cm and a central band of 1 MHz was used. The measurement vessel was a polystyrene cuvette having an optical path length of 0.1 cm and a sample volume of about 200 μL. The measurement vessel and the piezoelectric element were immersed in a glass vessel filled with water, and the space between the measurement vessel and the piezoelectric element was set to 2.5 cm. For the high speed preamplifier for amplifying the photoacoustic signal intensity, an ultrasonic wave pre-amplifier (Model 5682, manufactured by Olympus Corporation) having an amplification degree of +30 dB was used. The signal amplified was input into a digital oscilloscope (DPO4104, manufactured by Tektronix Inc.). The polystyrene cuvette was irradiated with pulse laser light from the outside of the glass vessel. A part of the scattering light here generated was detected by a photodiode and input into the digital oscilloscope as a trigger signal. The digital oscilloscope was set to a 32-run average display mode, and the measurement of the photoacoustic signal intensity averaged over 32 runs of the laser pulse irradiation was performed.

(Calculation of Photoacoustic Signal Intensity Per 100-nm-Equivalent Particle)

The particle dispersion was lyophilized to thereby calculate the weight concentration of a solid component included in the dispersion . . . . (a)

The density of each constituent material was assumed to be (g/cm³), and the weight per particle was calculated from the particle size of each particle . . . . (b)
The weight concentration determined in (a) was divided by the weight per particle determined in (b) to calculate the particle concentration in the particle dispersion . . . . (c)
The photoacoustic signal intensity per particle was calculated from the result of the photoacoustic signal measurement and the result of (c). Thereafter, when a 100-nm particle was present in the same composition, the respective values were calculated, being assumed to be in proportion to the volume ratio.

### Example 1-1

(Preparation 1 of Lipid Particle Containing Silicon Naphthalocyanine)

DSPC (61.2 mg), 20.4 mg of DSPE-PEG-OMe and 20.4 mg of cholesterol were dissolved in 1 mL of chloroform. Silicon 2,3-naphthalocyanine bis(trihexylsilyloxide) (hereinafter, sometimes abbreviated as Compound 1) (13 mg) was dissolved in 0.3125 mL of chloroform (hereinafter, the solution was sometimes abbreviated as solution of Compound 1). Above-described 1 mL of the chloroform solution, in which DSPC and the like were dissolved, and the total amount of solution of Compound 1 were loaded in a recovery flask and mixed, the solvent was distilled off at 40° C. under reduced pressure (Rotavapor R-205, manufactured by Buchi), and then vacuum drying (Vacuum oven VOS-301SD, manufactured by EYELA) was performed overnight. Ten mM HEPES solution (pH 7.3) (hereinafter, referred to as HEPES solution) (2.5 mL) was added to the resulting dried solid product of the lipid and Compound 1, and ultrasonic irradiation (three-frequency ultrasonic cleaner VS-100III, As One Corporation) was performed at 60° C. for 30 minutes. Thereafter, the mixture was diluted with the HEPES solution and then filtrated by a 0.45-μm filter, the resultant was centrifuged at room temperature 20000×g for 15 minutes, and the precipitate and the supernatant were recovered. The amounts of Compound 1 and solution of Compound 1 were changed as shown in Table 1 and 4 types (8 samples) of lipid particles were produced. Table 1 also shows the name, the particle size, the dye content, the wavelength a, the wavelength b, the absorption coefficient ratio, and the photoacoustic signal per 100-nm-equivalent particle of each sample.

TABLE 1

| | | | | | | | | | Photoacoustic |
|---|---|---|---|---|---|---|---|---|---|
| | | | | | | | | | signal |
| | | Amount of | | | | | | | per 100-nm |
| | Amount of | solution of | | Particle | Dye | | | Absorption | equivalent |
| Sample | Compound 1 | Compound 1 | Centrifugation | size | content | Wavelength a | Wavelength b | coefficient | particle |
| name | (mg) | (ml) | fraction | (nm) | (%) | (nm) | (nm) | ratio | (V/J/M) |
| 1-1-1 | 13.0 | 0.3125 | Supernatant | 86.1 | 0.07 | 782 | 737 | 4.1 | 1.8E+09 |
| 1-1-2 | | | Precipitate | 122.8 | 0.95 | 788 | 750 | 2.4 | 1.2E+10 |
| 1-2-1 | 26.0 | 0.625 | Supernatant | 87.3 | 0.07 | 782 | 737 | 3.7 | 1.5E+09 |
| 1-2-2 | | | Precipitate | 127.1 | 1.2 | 789 | 747 | 2.2 | 1.1E+10 |
| 1-3-1 | 51.9 | 1.25 | Supernatant | 89.7 | 0.04 | 781 | 737 | 4.3 | 9.7E+08 |
| 1-3-2 | | | Precipitate | 130.1 | 1.3 | 791 | 745 | 2.2 | 1.2E+10 |
| 1-4-1 | 103.9 | 2.5 | Supernatant | 90.0 | 0.05 | 780 | 737 | 4.3 | 4.9E+08 |
| 1-4-2 | | | Precipitate | 133.7 | 1.3 | 792 | 743 | 2.1 | 1.5E+10 |

### Example 1-2

### Relationship Between Dye Content and Absorption Coefficient Ratio

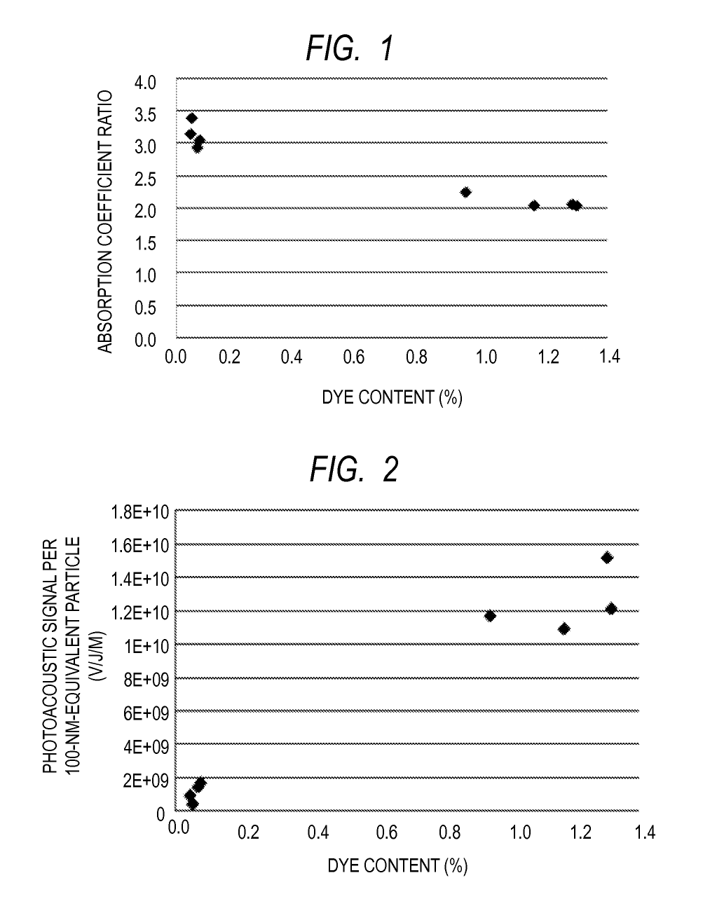

The relationship between the dye content and the absorption coefficient ratio of the lipid particle obtained in Example 1 is illustrated in FIG. 1. As illustrated in FIG. 1, the particle higher in dye content is lower in absorption coefficient ratio. The absorption coefficient ratio of the liposome described in Br. J. Cancer (1990), 62, 966-970 is about 7.3, and it is thus considered that the lipid particle obtained in Example 1 has an improved dye content as compared with the liposome described in Br. J. Cancer (1990), 62, 966-970.

### Example 1-3

### Relationship Between Dye Content and Photoacoustic Signal Per Particle

The relationship between the dye content and the photoacoustic signal per particle is illustrated in FIG. 2.

As illustrated in FIG. 2, it has been found that when the dye content is increased, the photoacoustic signal per particle is improved.

From the foregoing, it has been indicated that the lipid particle of the present Example, having a small absorption coefficient ratio, has a high dye content and furthermore, when the absorption coefficient is 2.5 or less, the dye content is high and the photoacoustic signal is as very high as 1.0E+10 or more.

### Example 2-1

### Preparation 2 of Lipid Particle Containing Silicon Naphthalocyanine

DSPC (61.2 mg), 20.4 mg of DSPE-PEG-OMe and 1.7 mg of cholesterol were dissolved in 1 mL of chloroform. Silicon 2,3-naphthalocyanine bis(trihexylsilyloxide) (hereinafter, sometimes abbreviated as Compound 1) (1.3 mg) was dissolved in 1 mL of chloroform (hereinafter, the solution was sometimes abbreviated as solution of Compound 1). Above-described 1 mL of the chloroform solution, in which DSPC and the like were dissolved, and the total amount of solution of Compound 1 were loaded in a recovery flask and mixed. The solvent was distilled off at 40° C. under reduced pressure (Rotavapor R-205, manufactured by Buchi), and then vacuum drying (Vacuum oven VOS-301SD, manufactured by EYELA) was performed overnight. A PBS solution (2.5 mL) was added to the resulting dried solid product of the lipid and Compound 1, and ultrasonic irradiation (three-frequency ultrasonic cleaner VS-100III, As One Corporation) was performed at 60° C. for 30 minutes. The mixture was filtrated by a 0.22-μm filter, the resultant was then centrifuged at room temperature 288000×g for 17 minutes, and the precipitate was recovered to provide lipid particle 2-1.

(Confirmation of Tumor Accumulation Property of Lipid Particle 2-1 Containing Silicon Naphthalocyanine)

In confirmation of the tumor accumulation property, a female outbred BALB/c Slc-nu/nu mouse (six-week old at the time of purchase) (Japan SLC, Inc.) was used. For 1 week before the mouse was allowed to bear cancers, the mouse was habituated through the use of a standard diet and bed under such an environment that the diet and drinking water were available ad libitum. Colon26 (mouse colon cancer cell) was subcutaneously injected to the mouse. All tumors were fixed until the experiment, and the body weight of the mouse was 17 to 22 g. One hundred μL (13 nmol as the dye) of the particle dispersion was intravenously injected to the caudal portion of the mouse allowed to bear cancers.

Then, the mouse to which the particle dispersion was administered was euthanized at 24 hours after the administration, and then colon26 tumors were extirpated. The tumor tissue was transferred to a plastic tube, and then homogenized by adding an aqueous 1% Triton-X100 solution in an amount 1.25 times as large as the weight of the tumor tissue. Then, tetrahydrofuran (THF) was added in an amount 20.25 times as large as the weight of the tumor tissue. Odyssey (registered trademark) CLx Infrared Imaging System was used to measure the fluorescence intensity of the homogenate solution, quantifying the dye amount in the tumor tissue.

Table 2 shows the particle size, the dye content, the wavelength a, the wavelength b, the absorption coefficient ratio, the photoacoustic signal per 100-nm-equivalent particle, and the tumor accumulation property of lipid particle 2-1.

TABLE 2

| | | | | | | Photo- | |
|---|---|---|---|---|---|---|---|
| | | | | | | acoustic | |
| | | | | | | signal | |
| | | | | | Ab- | per | |
| | | | | | sorp- | 100-nm | Tumor |
| | Par- | | Wave- | Wave- | tion | equiv- | accumu- |
| | ticle | Dye | length | length | coeffi- | alent | lation |
| Sample | size | content | a | b | cient | particle | property |
| name | (nm) | (%) | (nm) | (nm) | ratio | (V/J/M) | (% ID/g) |
| 2-1 | 92.3 | 0.21 | 777 | 735 | 3.6 | 5.5E+09 | 10.9 |

It has been indicated that the resulting particle is high in photoacoustic signal per 100-nm-equivalent particle and high in tumor accumulation property.

The lipid particle according to the present invention is high in photoacoustic signal intensity.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-131878, filed Jun. 24, 2013, which is hereby incorporated by reference herein in its entirety.

What is claimed is:

1. A photoacoustic contrast agent having a lipid particle containing a silicon naphthalocyanine analog, wherein an absorption coefficient A that is the largest value and an absorption coefficient B that is the second largest value and a local maximum value in a wavelength region of 700 nm to 800 nm, of the lipid particle, satisfy the following expression (1):
0<A/B≦5.0 (1).

2. The photoacoustic contrast agent according to claim 1, wherein the absorption coefficient A is an absorption coefficient at a first wavelength a that is an absorption local maximum wavelength of a monomer of the silicon naphthalocyanine analog, and the absorption coefficient B is an absorption coefficient at a second wavelength b that is an absorption local maximum wavelength of an aggregate of the silicon naphthalocyanine analog.

3. The photoacoustic contrast agent according to claim 1, wherein the first wavelength a is in a range of more than 750 nm and 800 nm or less.

4. The photoacoustic contrast agent according to claim 1, wherein the second wavelength b is in a range of 700 nm or more and 750 nm or less.

5. The photoacoustic contrast agent according to claim 1, wherein the absorption coefficient A and the absorption coefficient B satisfy the following expression (2):
0<A/B≦2.5 (2).

6. The photoacoustic contrast agent according to claim 1, wherein the lipid particle has phospholipid.

7. The photoacoustic contrast agent according to claim 1, wherein the phospholipid is at least one selected from the group consisting of distearoylphosphatidylcholine, dimyristoylphosphatidylcholine, dipalmitoylphosphatidylcholine, dioleylphosphatidylcholine, distearoylphosphatidylserine, distearoylphosphatidylglycerol and dipalmitoylphosphatidic acid.

8. The photoacoustic contrast agent according to claim 1, wherein the lipid particle has a liposome.

9. The photoacoustic contrast agent according to claim 1, wherein a structure of the silicon naphthalocyanine analog is represented by the following chemical formula (1): 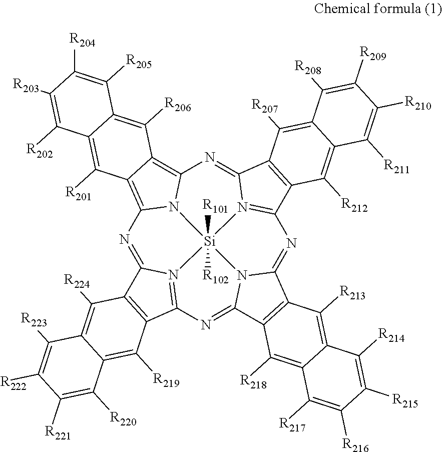
wherein R₂₀₁, R₂₀₂, R₂₀₃, R₂₀₄, R₂₀₅, R₂₀₆, R₂₀₇, R₂₀₈, R₂₀₉, R₂₁₀, R₂₁₁, R₂₁₂, R₂₁₃, R₂₁₄, R₂₁₅, R₂₁₆, R₂₁₇, R₂₁₈, R₂₁₉, R₂₂₀, R₂₂₁, R₂₂₂, R₂₂₃ and R₂₂₄ may be each the same or different, and each represent a hydrogen atom, a halogen atom, an acetoxy group, an amino group, a nitro group, a cyano group, or an alkyl group having 1 to 18 carbon atoms or an aromatic group that is unsubstituted or substituted with one or more functional groups selected from the group consisting of a halogen atom, an acetoxy group, an amino group, a nitro group, a cyano group or an alkyl group having 1 to 18 carbon atoms;
and, R₁₀₁ and R₁₀₂ may be each the same or different, and each represent —OH, —OR₁₁, —OCOR₁₂, —OSi(—R₁₃)(—R₁₄)(—R₁₅), a halogen atom, an acetoxy group, an amino group, a nitro group, a cyano group, or an alkyl group having 1 to 18 carbon atoms or an aromatic group that is unsubstituted or substituted with one or more functional groups selected from the group consisting of a halogen atom, an acetoxy group, an amino group, a nitro group, a cyano group or an alkyl group having 1 to 18 carbon atoms;
wherein R₁₁, R₁₂, R₁₃, R₁₄ and R₁₅ may be each the same or different, and each represent one that is unsubstituted or substituted with one or more functional groups selected from the group consisting of a halogen atom, an acetoxy group, an amino group, a nitro group, a cyano group or an alkyl group having 1 to 18 carbon atoms.

10. The photoacoustic contrast agent according to claim 1, wherein the silicon naphthalocyanine analog has silicon 2,3-naphthalocyanine bis(trihexylsilyloxide).

11. The photoacoustic contrast agent according to claim 1, wherein the lipid particle has polyethyleneglycol.

12. The photoacoustic contrast agent according to claim 11, wherein the polyethyleneglycol has a molecular weight of 2000 or more and 100000 or less.

13. The photoacoustic contrast agent according to claim 11, wherein a proportion of the polyethyleneglycol in the lipid particle is 0.1% by mol or more and 10% by mol or less.

14. The photoacoustic contrast agent according to claim 1, wherein the lipid particle further has cholesterol.
